(19) United States
(12) Patent Application Publication
(10) Pub. No.: US 2016/0235515 A1
(43) Pub. Date: Aug. 18, 2016

(54) Embolic Protection Device

(71) Applicant: Innovative Cardiovascular Solutions, LLC, Kalamazoo, MI (US)

(72) Inventor: William M. Merhi, Grand Rapids, MI (US)

(21) Appl. No.: 15/030,431

(22) PCT Filed: Oct. 21, 2014
(86) PCT No.: PCT/US2014/061504

§ 371 Date: Apr. 19, 2016

Related U.S. Application Data
(60) Provisional application No. 61/893,331, filed on Oct. 21, 2013.

(51) Int. Cl. A61F 2/01

(52) U.S. Cl. CPC A61F 2/013; A61F 2/2433

(57) ABSTRACT

The present invention includes an embolic protection device comprising a catheter having a self-expanding embolic filter that is disposed around the catheter proximal to a distal portion, wherein the embolic filter comprises a frame that has at least two lobes, and the frame defines an opening of the embolic filter that faces the distal end of the catheter; and a deployment mechanism that is disposed around at least a portion of the catheter, wherein the deployment mechanism is longitudinally movable with respect to the catheter, the deployment mechanism is configured to contain the embolic filter in a collapsed configuration, and the embolic filter is configured to self-expand upon the longitudinal retraction of the deployment mechanism.

### CROSS REFERENCE TO RELATED APPLICATION

This PCT application claims the benefit of U.S. provisional application No. 61/893,331, filed on Oct. 21, 2013. This document is incorporated herein by reference

### TECHNICAL FIELD OF THE INVENTION

This application relates to embolic protection devices and closed-heart surgical procedures using these devices.

### BACKGROUND OF THE INVENTION

During percutaneous cardiac procedures, precise positioning of various instruments and devices can be important. For example, when performing a percutaneous valve replacement procedure, the valve is generally placed no more than 4-6 millimeters (mm) below the lower border of the aortic annulus. Placing the valve prosthesis too low or too high can result in severe leaking of the valve, which in some cases can be fatal. Therefore, it can be important to identify the lower border of the annulus to use as a reference point. A pigtail catheter may be used to inject a contrast agent to allow for visualization for proper positioning. Pigtail catheters may include a coiled distal portion and a plurality of small holes in the catheter side walls. The small holes allow for the introduction of contrast materials into the body for imaging purposes or drainage of fluids from the body. The coiled distal portion helps hold the catheter in place and can slow the flow of contrast fluids from the catheter lumen to avoid causing internal injuries or poor imaging results.

A potential complication of cardiac procedures such as valve replacement and repair is that plaque, calcium, and/or thrombi in the vessels, valves, and/or cardiac chambers can be dislodged and cause an embolism. Approximately 2.9%-6.7% of patients undergoing transfemoral transcatheter aortic-valve implantation (TAVI) have a stroke within 30 days, and even more (4.5%-10.6%) have a stroke within a year, often leading to death. Furthermore, up to 85% of patients undergoing TAVI have evidence of embolic phenomenon to the brain based on neuroimaging studies. Although clinically silent, it can be associated with cognitive decline (Astraci 2011; Ghanem 2010; Kahlert 2010; Rodes-Caban 2011). There are a few devices on the market designed to protect the brain, abdominal organs, and carotid arteries from emboli; however, these devices have various disadvantages. For example, the Embrella Embolic Deflector®, available from Edwards Lifesciences of Irvine, California, deflects emboli from the carotid arteries into the descending aorta, but does not trap the emboli, so there is a risk of embolisms in other areas of the body. The EMBOL-X®, also available from Edwards Lifesciences, employs a filtering screen, but it is designed for use in open heart procedures, which present additional medical risks and increased morbidity. Additionally, the use of multiple devices, for example a catheter for visualization and a separate filter device, lengthens the procedure time and increases the risk of complications to the patient.

### SUMMARY OF THE INVENTION

These and other needs are met by the present invention, which is directed to an embolic protection device comprising a deployable embolic filter that is disposed around a catheter having a distal portion that can assume an arcuate configuration being at least a semi-circle.

The combination of the catheter and the embolic filter in the same device may provide the benefits of both devices individually, as well as provide a synergistic effect. For example, the integration of the catheter and the embolic filter can decrease the duration of the medical procedure and reduce complications. In other examples, the expansion of the embolic filter may help to anchor the catheter into position to provide a more accurate position of the catheter than if the position of the catheter could be influenced by blood flow, tissue movement, and the like. In a valve replacement procedure, anchoring of the catheter and more accurate positioning of the catheter may in turn help ensure that the valve prosthesis is properly positioned and stabilized. For another example, the position of the catheter may ensure that the filter is being properly positioned.

In some aspects, the embolic protection device comprises a multi-lobed self-expanding embolic filter having two or more lobes that is coupled to a catheter and an outer sheath movable with respect to the embolic filter and the catheter. The outer sheath holds the embolic filter in a collapsed configuration when surrounding the embolic filter and is proximally retracted to deploy the embolic filter. The outer sheath may recapture the embolic filter and any debris captured therein by being distally advanced. The filter and outer sheath might both be movable with respect to the catheter, for example to be able to move the embolic filter longitudinally without having to move the entire catheter longitudinally. An embolic filter comprising two or more lobes is advantageous because of the ease in manufacturing and its ability to more fully engage the body lumen when in the expanded configuration.

In some aspects, the catheter has a proximal end and a distal end. A lumen extends from the proximal end of the catheter to the distal end of the catheter. In some embodiments, the lumen may be configured to house a guidewire.

In some aspects, the catheter is a pigtail catheter. A pigtail catheter is configured to curl at the distal end of the catheter, forming a generally arcuate shape that is at least a semi-circle. The pigtail may have a radiopaque marker viewable on x-rays or other medical imaging devices. The radiopaque marker is on the distal section of the curled pigtail in the form of a longitudinal marker, multiple bands, or the like. The pigtail may additionally have one or more apertures to dispense drugs and/or contrast agents through the lumen

In some aspects, a guidewire is inserted through the patient's skin and into a body lumen such as a femoral, radial, or brachial artery and steered near a target site. The guidewire is inserted into a lumen of the embolic protection device, and the embolic protection device is pushed or tracked over the guidewire to the target site. When the guidewire is retracted from at least the distal portion of the catheter, the catheter assumes a generally arcuate shape. The radiopaque marker on the catheter is used to visualize and position the catheter. Once the catheter is in position, the outer sheath is retracted to deploy the embolic filter across the vessel. The user can then perform a procedure such as valve replacement, valve repair, radio frequency ablation, and the like. When the procedure is completed, the outer sheath is advanced to recapture the embolic filter and any debris trapped in the embolic filter. The device is then retracted, with the catheter being atraumatic to vessels during retraction.

Another aspect is a method of capturing embolic debris during a closed-heart surgical procedure comprising inserting the distal end of the catheter of the embolic protection device into a body lumen. The method further comprises allowing the multi-lobed embolic filter to assume an expanded, deployed configuration having a distal opening that spans the body lumen.

### BRIEF DESCRIPTION OF THE FIGURES

The following figures are provided by way of example and are not intended to limit the scope of the claimed invention.

FIGS. 1A-1B show partial side views of one embodiment of an embolic protection device.

FIG. 1C shows a transverse cross sectional view of one embodiment of a multi-lobed embolic filter.

FIG. 1D shows a partial side view of one embodiment of a frame of a multi-lobed embolic filter.

FIGS. 2A-2B show partial side views of one embodiment of an embolic protection device.

FIGS. 3A-3D show partial side views of one embodiment of an embolic protection device.

FIGS. 4A-4C show partial side views of one embodiment of an embolic protection device.

FIGS. 5A-5D show one embodiment of a method of capturing embolic debris using an embolic protection device.

FIG. 6 shows one embodiment of a method of deflecting and capturing embolic debris using an embolic protection device.

FIG. 7 shows one embodiment of a method of deflecting and capturing embolic debris using an embolic protection device.

### DETAILED DESCRIPTION OF THE INVENTION

The present invention is directed to embolic protection devices and methods of capturing embolic debris during surgical procedures.

### I. Definitions

As used herein, the term “closed-heart” refers to any surgical procedure involving the heart, wherein the chest cavity is not opened.

As used herein, the term “woven” refers to any material that comprises a plurality of strands, wherein the strands are interlaced to form a net, mesh, or screen. Without limitation, examples of woven materials include netting or mesh comprising a polymer, metal, or metal alloy.

As used herein, the term “non-woven” refers to any material that comprises a continuous film. Non-woven material may be permeable, semi-permiable, or non-permeable. For example, permeable or semi-permeable non-woven material may optionally include one or more pores through which a fluid may pass.

As used herein, the term “alloy” refers to a homogenous mixture or solid solution produced by combining two or more metallic elements, for example, to give greater strength or resistance to corrosion. For example, alloys include brass, bronze, steel, nitinol, chromium cobalt, MP35N, 35NLT, elgiloy, and the like.

As used herein, “nitinol” and “nickel titanium” are used interchangeably to refer to an alloy of nickel and titanium.

As used herein, “chromium cobalt” refers to an alloy of chromium and cobalt.

As used herein, “MP35N” refers to an alloy of nickel and cobalt.

As used herein, “35NLT” refers to a cobalt-based alloy that may also comprise chromium, nickel, molybdenum, carbon, manganese, silicon, phosphorum, sulphur, titanium, iron, and boron.

As used herein, “elgiloy” refers to an alloy of cobalt, chromium, nickel, iron, molybdenum, and manganese.

As used herein, a “body lumen” refers to the inside space of a tubular structure in the body, such as an artery, intestine, vein, gastrointestinal tract, bronchi, renal tubules, and urinary collecting ducts. In some instances, a body lumen refers to the aorta.

### II. Embolic Protection Devices

Although certain embodiments and examples are described below, those of skill in the art will appreciate that the disclosure extends beyond the specifically disclosed embodiments and/or uses and obvious modifications and equivalents thereof. Thus, it is intended that the scope of the disclosure herein disclosed should not be limited by any particular embodiments described below.

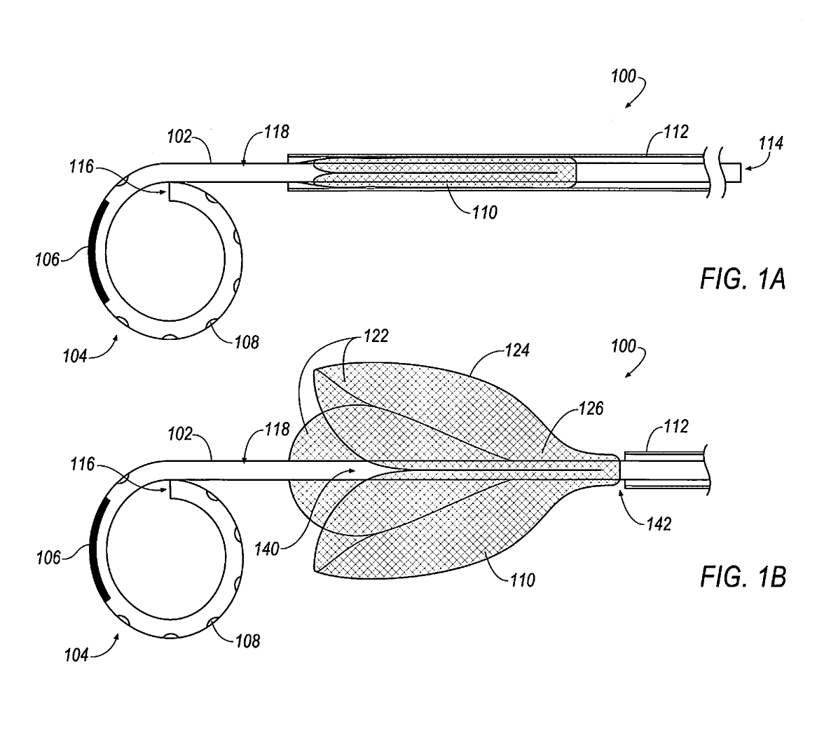

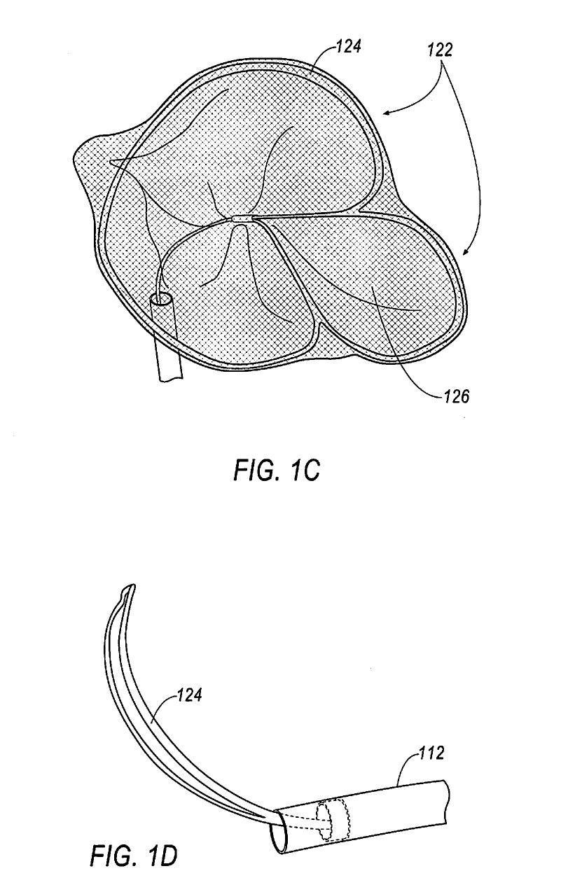

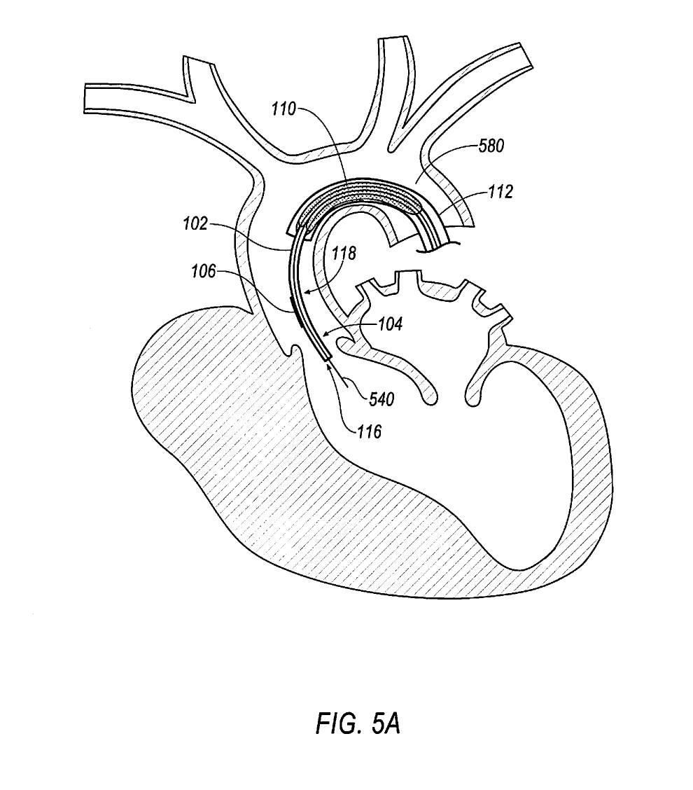

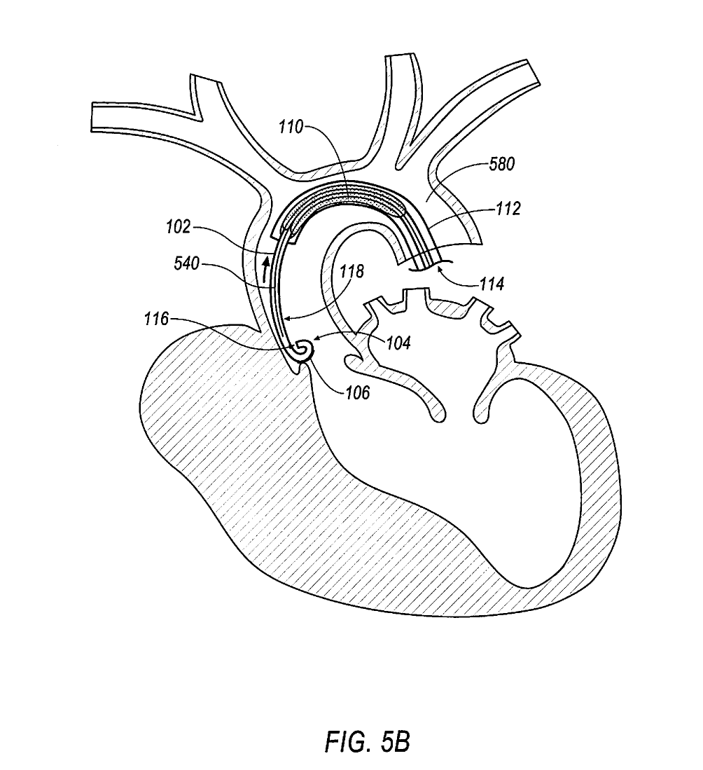

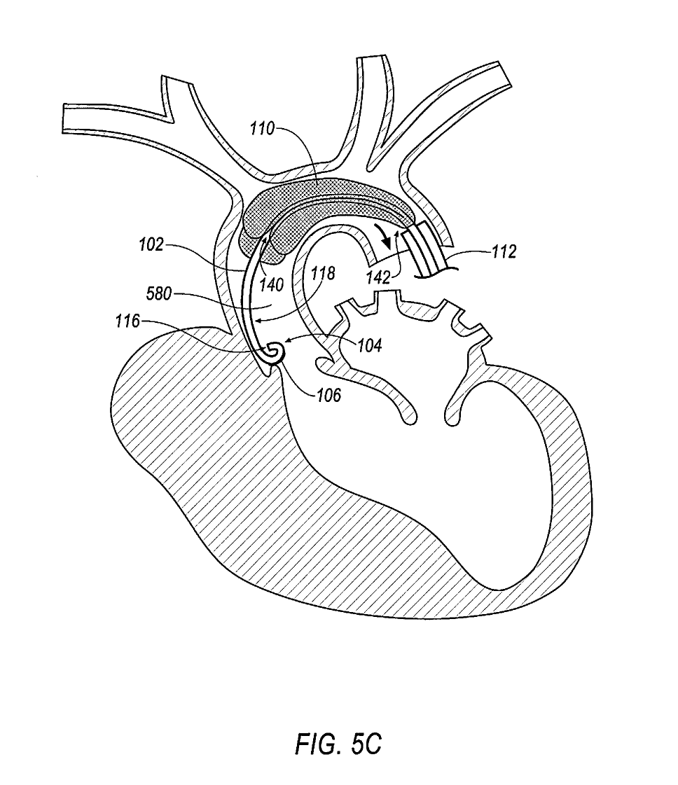

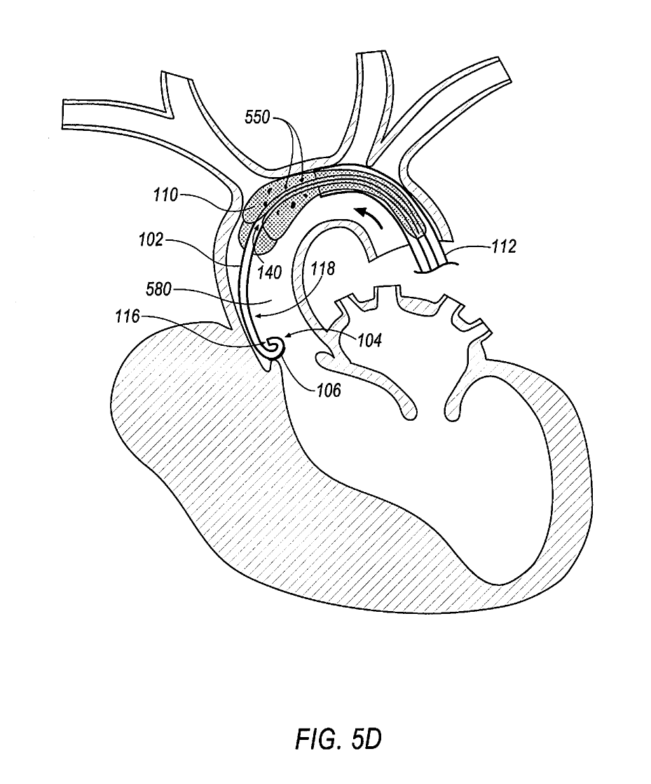

FIGS. 1A and 1B illustrate embodiments of an embolic protection device 100. The device 100 comprises a catheter 102 (e.g., a pigtail catheter) having a proximal end 114, a distal end 116, and a lumen 118 extending from the proximal end 114 to the distal end 116. The lumen 118 may be configured to house a guidewire 540 (FIGS. 5A and 5B). The catheter 102 includes a distal portion 104 configured to assume a generally arcuate shape being at least a semi-circle. A side wall of the catheter 102 may optionally include one or more apertures 108 in the distal portion 104 that are configured to deliver one or more fluids (e.g., an imaging dye, oxygenated blood, saline, any combination thereof, or the like) to a body lumen 580 (see FIG. 5). The apertures 108 (the plural intended to include embodiments in which the distal portion includes one aperture 108) are in fluid communication with the lumen 118. The distal portion 104 of the catheter 102 includes a longitudinally-extending radiopaque marker 106 that is configured to be arcuate when the distal portion 104 is in the generally arcuate shape. The device 100 further comprises a multi-lobed self-expanding embolic filter 110 and a deployment device 112 (e.g., a longitudinally retractable outer sheath or a longitudinally retractable ring). The embolic filter 110 is disposed around the catheter 102. As shown in FIG. 1B, in its expanded configuration, the embolic filter 110 includes a distal opening 140 that is defined by the frame, faces the distal end 116 of the catheter, and extends proximally from the distal opening 140 to a closed proximal end 142. The multi-lobed embolic filter 110 allows the distal opening 140 of the embolic filter 110 to engage at least a portion of the interior body lumen 580 (see FIG. 5) wall.

FIG. 1C is a cross-sectional view of the distal opening 140 of the embolic filter 110 when the embolic filter assumes an expanded configuration. The embolic filter 110 has at least two lobes 122, which are disposed around the catheter 102 in a generally conical shape from the distal opening 140 to the closed proximal end 142 when the embolic filter 110 is in its expanded configuration. The lobes are defined by a frame 124 and include a filter medium 126 that is supported by and attached to at least a portion of the frame 124. The embolic filter 110 is coupled (e.g., by adhering, welding, soldering, coupling using a separate component, combinations thereof, and the like) to the catheter 102, so that the lobes 122 of the embolic filter 110 are disposed around the catheter. In some embodiments, the distal opening 140 of the embolic filter 110 has a diameter of from about 2 cm to about 6 cm (e.g., from about 2.5 cm to about 5 cm or about 4.5 cm). The embolic filter 110 can comprise any suitable size or diameter to accommodate anatomic variability in patients' body lumens 580 (see FIG. 5). In some embodiments, the embolic filter 110 is coupled to the catheter 102 along the entire length of the embolic filter 110. In some embodiments, the embolic filter 110 is coupled to the catheter 102 at the proximal and/or distal ends of the embolic filter 110 and/or at any other points there between.

FIG. 1D illustrates only the frame 124 of the embolic filter 110. In some embodiments, the frame 124 comprises a shape memory material (e.g., a metal alloy or polymer). Examples of shape memory materials include, without limitation, nitinol, chromium cobalt, and/or other metal alloys such as MP35N, 35NLT, elgiloy, and the like. In some embodiments, the frame 124 is laser cut from a tube or a sheet. In some embodiments, the frame 124 may be configured so that it forms a generally arcuate shape.

In some embodiments, the filter medium 126 comprises a braided mesh, for example braided nitinol mesh. In some embodiments, the filter medium 126 comprises a porous membrane, for example a semi-permeable polyurethane membrane. In other embodiments, the filter has a pore size of from about 100 microns to about 150 microns (e.g., about 125 microns).

In some embodiments, the embolic filter 110 comprises an anti-thrombogenic coating (e.g., a heparin coating or other coating comprising a thrombin or platelet inhibitor) to advantageously reduce thrombogenicity.

The embolic filter 110 is configured to self-expand to a radially expanded configuration, shown in FIGS. 1B and 1C, when not confined by the deployment device (e.g., outer sheath 112).

In some embodiments wherein the deployment mechanism comprises the outer sheath 112, the outer sheath 112 is configured to be circumferentially disposed around at least a portion of the catheter 102 and the embolic filter 110. The outer sheath 112 is configured to contain or house the embolic filter 110 in a collapsed configuration. The outer sheath 112 is longitudinally movable with respect to the catheter 102, and can be longitudinally retracted, i.e., moved longitudinally in a proximal direction, to deploy the embolic filter 110 and longitudinally advanced, i.e., moved longitudinally in a distal direction, to recapture the embolic filter 110 and any embolic material collected by the embolic filter 110. The embolic filter 110 is configured to self-expand upon longitudinal retraction of the outer sheath. A device according to the disclosure herein can comprise some or all of the features of the embolic protection device 100 shown in FIGS. 1A-1B, and is described herein in various combinations and subcombinations. In some embodiments, the embolic filter 110 is configured to at least partially collapse upon longitudinal extension of the outer sheath. In these embodiments, the distal opening 140 assumes a substantially closed configuration thereby sequestering or substantially sequestering the filtered material.

The catheter 102 may comprise a flexible material so as to be maneuverable within a body lumen 580 (see FIG. 5) as described herein. For example, in some embodiments, the catheter 102 comprises a metal or metal alloy. In other embodiments, the catheter 102 comprises a polymer (e.g., polyurethane, silicone, latex, polytetrafluoroethylene (PTFE), a plastic material, any combination thereof, or the like). In some embodiments, the catheter 102 comprises a metal-reinforced plastic (e.g., including nitinol, stainless steel, and the like). Other materials are also possible. In some embodiments, the catheter 102 is substantially free of latex (natural or synthetic), which may cause allergic reactions in some patients. In some embodiments, the catheter 102 comprises braid-reinforced tubing to advantageously increase the strength of the catheter 102. In some embodiments, the catheter 102 comprises a braided catheter shaft including a layer of braided wire between two layers of catheter tubing, which may increase the strength of the catheter 102. In some embodiments, the catheter 102 does not include a braided layer, which may increase the flexibility of the catheter 102. In some embodiments, the catheter 102 comprises a lubricious coating, for example a coating having a low friction coefficient, to advantageously allow for smoother navigation through tortuous vasculature. In some embodiments, the catheter 102 coating has anti-thrombotic properties to advantageously inhibit thrombus formation. In some embodiments, the catheter 102 has a size (i.e., outside diameter) between about 3 French and about 5 French (between about 2 mm and about 3 mm). Other sizes are also possible, for example depending on the size of the target body lumen 580 (see FIG. 5) of a particular patient. In some embodiments, the catheter 102 has a length between about 65 centimeters (cm) and about 135 cm. Other lengths are also possible, for example to allow for insertion of the catheter 102 in the femoral, radial, brachial, or subclavian artery. The catheter 102 can be manufactured, for example, by extrusion, injection molding, or another suitable process.

The radiopaque marker 106 extends longitudinally along a section of the distal portion 104 of the catheter 102. When the distal portion 104 assumes the generally arcuate shape, the radiopaque marker 106 is also generally arcuate. In some embodiments, the radiopaque marker is located on a distal-most section of the catheter 102. In some embodiments, the radiopaque marker 106 has a length of about 1 cm. The radiopaque marker 106 comprises a radiopaque material, for example platinum, tantalum, tungsten, palladium, and/or iridium. Other radiopaque materials are also possible. In some embodiments, a material may be considered radiopaque, for example, if the average atomic number is greater than 24 or if the density is greater than about 9.9 g/cm³.

The outer sheath 112 comprises a hollow tube configured to circumferentially surround at least a portion of the catheter 102. The outer sheath 112 is longitudinally movable with respect to the catheter 102 and is configured to at least partially contain or house the embolic filter 110 in a collapsed configuration when circumferentially surrounding the embolic filter 110, for example, as shown in FIG. 1A. The outer sheath 112 is longitudinally proximally retractable to release the embolic filter 110 to the expanded, open configuration when not contained by the outer sheath 112. In some embodiments, the outer sheath 112 extends proximally to the proximal end 114 of the catheter 102 so that the user can grasp and manipulate the outer sheath 112 directly. In some embodiments, the outer sheath 112 extends proximally over only a portion of the catheter 102, and a secondary device (e.g., a push-rod such as found in stent deployment systems) is coupled to the outer sheath 112 (e.g., to the proximal end of the outer sheath 112) to allow for indirect manipulation of the outer sheath 112. Manipulation of the outer sheath 112 may be mechanical, electronic, manual, combinations thereof, and the like.

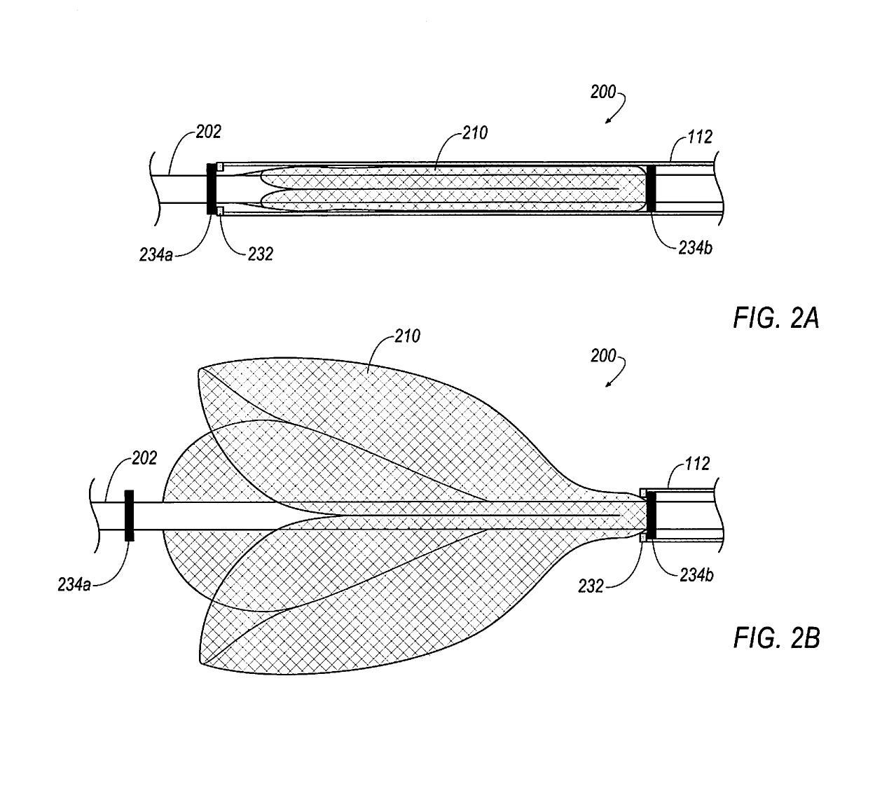

FIGS. 2A and 2B illustrate embodiments of an alternative deployment mechanism for an embolic protection device 200 comprising a catheter 202, an embolic filter 210, and a movable outer sheath 212. In some embodiments, the outer sheath 212 can include an optional lip 232 protruding inwardly from the distal end of the outer sheath 212. The catheter 202 can include one or more shoulders 234 (e.g., a distal shoulder 234a and a proximal shoulder 234b) protruding outwardly from an outer wall of the catheter 202. The lip 232 of the outer sheath 212 is configured to engage the shoulder or shoulders 234 of the catheter 202 to inhibit or prevent the outer sheath 212 from moving excessively in either the proximal or distal direction. The lip 232 and shoulder 234 may be arcuate, pronged, and combinations thereof, and the like.

In some embodiments, the outer sheath 212 and/or the catheter 202 comprise nubs and/or detents configured to provide information to the user about the longitudinal position of the outer sheath without inhibiting further movement. In some embodiments, the outer sheath 212 and the catheter 202 comprise lips 232, shoulders 234, and detents and nubs (e.g., to inhibit longitudinal movement of the outer sheath 212 excessively in either direction, and to provide information about the extent of movement of the outer sheath 212 relative to the catheter 202 (e.g., ½ retracted, ¼ retracted, etc.)).

Benefits of the outer sheath 212 deployment mechanism may include its simplicity, ease of operation, and small number of moving parts. The embolic protection device 200 is well-suited for use in conjunction with delicate cardiac procedures having serious risks. As the duration of the procedure increases, the risk of complications typically increases as well. Therefore, it can be advantageous that the user be able to quickly and easily deploy and recapture the embolic filter 210. A more complicated device could be more difficult to operate and could be more likely to malfunction or cause adverse effects. The ability to move the outer sheath 212 relative to the filter 210 can advantageously allow the user to partially recapture the embolic filter 210, for example to adjust the width of the distal opening 140. In some embodiments, narrowing the distal opening 140 allows the user to introduce a second catheter or instrument to the patient's body lumen 580 (see FIG. 5) and maneuver the second catheter or instrument around and past the catheter 202 and embolic filter 210, as described herein.

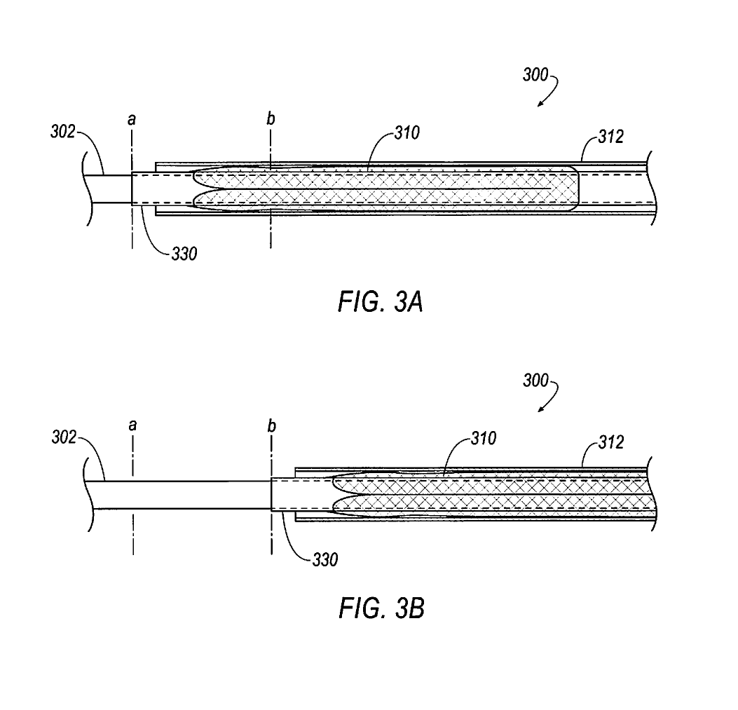

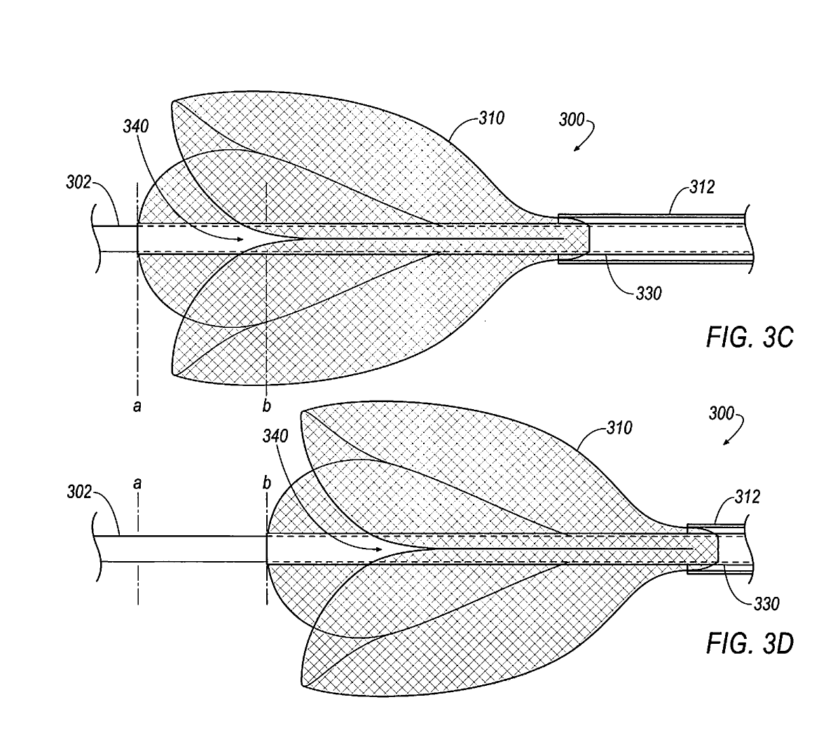

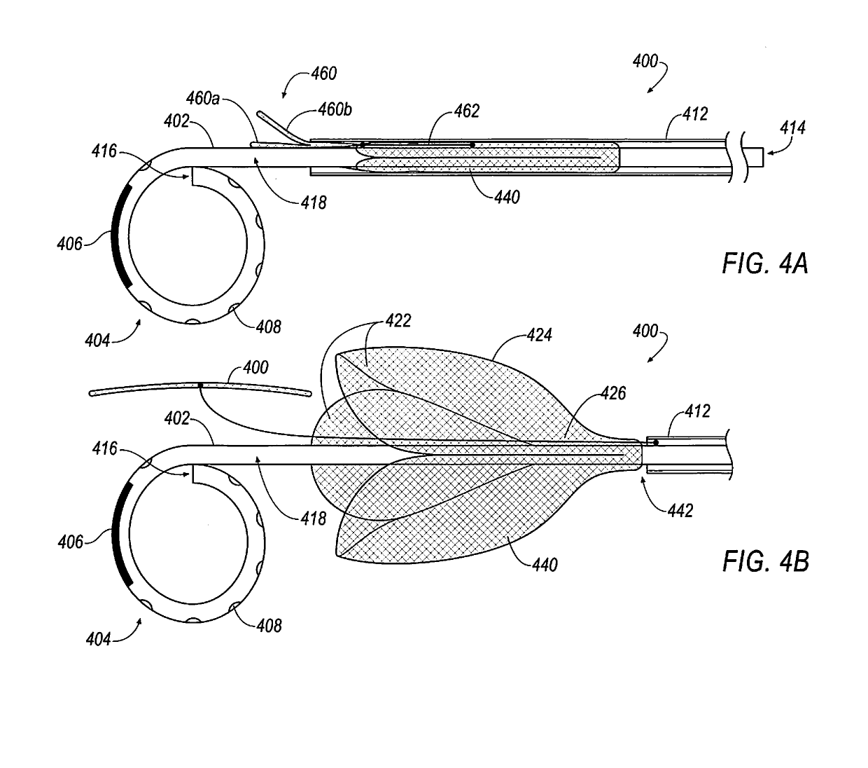

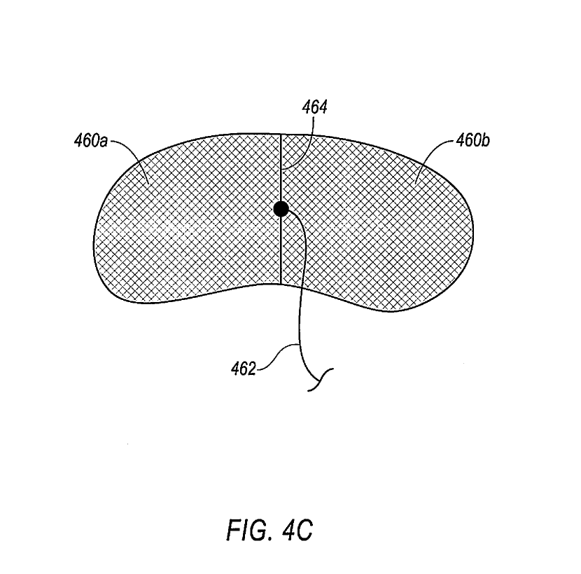

FIGS. 3A-4D illustrate embodiments of an embolic protection device 300 in which an embolic filter 310 is movably coupled to a catheter 302 and is longitudinally movable with respect to the catheter 302. In some embodiments, the embolic filter 310 is coupled to an intermediate tube 330 that at least partially circumferentially surrounds the catheter 302. The intermediate tube 330 is longitudinally movable with respect to the catheter 302. An outer sheath 312 is configured to at least partially circumferentially surround both the catheter 302 and the intermediate tube 330. The intermediate tube 330 and the outer sheath 312 can be moved simultaneously and independently. The longitudinal position of the embolic filter 310 with respect to the catheter 302 can be adjusted while the embolic filter 310 is in the collapsed configuration or in a deployed or partially deployed, expanded configuration. In some embodiments, the perimeter of the distal opening of the embolic filter 310 comprises one or more radiopaque markers to allow the user to visualize the position of the distal opening, for example, with respect to various anatomical landmarks. For example, if the user is performing a procedure on a patient's aortic valve and wants to prevent emboli from entering the cerebral arteries, the radiopaque markers can be used to ensure the distal opening of the embolic filter 310 is positioned in the ascending aorta upstream from the carotid arteries.

FIG. 3A shows the embolic filter 310 confined in a closed configuration by the outer sheath 312 and a distal end of intermediate tube 330 at position a. If the intermediate tube 330 is held stationary at position a, the outer sheath 312 can be retracted to deploy the embolic filter 310, as shown in FIG. 3C. If the intermediate tube 330 and outer sheath 312 are instead moved simultaneously, the embolic filter 310 remains confined by the outer sheath 312 while the longitudinal position of the embolic filter 310 is adjusted. For example, FIG. 3B shows the embolic filter 310 still confined by outer sheath 312, but the intermediate tube 330 has been retracted so that the distal end of the intermediate tube 330 is at position b. If the intermediate tube 330 is then held stationary at position b, the outer sheath 312 can be retracted to deploy the embolic filter 310, as shown in FIG. 3D. The intermediate tube 330 and outer sheath 312 can be moved to adjust the longitudinal position of the embolic filter 310 in a deployed or partially deployed configuration. For example, the intermediate tube 330 and outer sheath 312 can be moved simultaneously to retract the intermediate tube 330 from the position as shown in FIG. 3C to the position b as shown in FIG. 3D. When the embolic filter 310 is partially deployed, the embolic filter 310 may not be in contact with the vessel walls and freely movable, for example due to lack of wall apposition. When the embolic filter 310 is fully deployed, any debris dislodged during movement may be trapped in the embolic filter 310.

In addition to those described in detail herein, a wide variety of deployment mechanisms for embolic filters are possible. For example, a deployment system may comprise a portion of an annular sheath including inward end protrusions that are guided in tracks along the catheter body. Certain such embodiments may advantageously reduce the profile of the catheter. For another example, a deployment system may comprise a threaded sheath that longitudinally moves upon twisting by the user. For yet another example, a deployment system may comprise a plurality of annular bands that can capture the embolic filter longitudinally and/or circumferentially. Combinations of the deployment systems described herein and other deployment systems are also possible.

FIG. 4 shows another example embodiment of an embolic protection device 400 comprising a catheter 402, a deflector 460, an embolic filter 410, and a movable outer sheath 412. In some embodiments, the device 400 is similar to embolic protection device 100 with the addition of the deflector 460.

Various types and designs of deflectors can be used with an embolic protection device such as device 400. Such deflectors can have different shapes and/or sizes and can vary in where and how they are coupled to the catheter. For example, deflectors can be made in various sizes, for example to accommodate differences in patient anatomy. In some embodiments, the deflector comprises a shape memory material, for example including nitinol, chromium cobalt, and/or alloys such as MP35N, 35NLT, elgiloy, and the like. In some embodiments, the deflector comprises a porous membrane, for example a semi-permeable polyurethane membrane, mounted to a self-expanding frame, for example a frame comprising a shape memory material.

The example deflector 460 shown in FIGS. 4A-4C has a generally butterfly or elliptical shape with two wings or petals 460a and 460b extending to either side of a central axis 464. The wings 460a and 460b may be the same or different in size shape, material, and the like. The deflector 460 is coupled to a side of the catheter 402 via an elongate member 462 that is coupled (e.g., by adhering, welding, soldering, coupling using a separate component, combinations thereof, and the like) at one end to the central axis 464 of the deflector 460 and at the other end to the catheter 402. In some embodiments, the elongate member 462 comprises a shape memory material, for example including nitinol, chromium cobalt, and/or alloys such as MP35N, 35NLT, elgiloy, and the like that is configured (e.g., shape set) to bias the deflector away from the catheter 402. The deflector 460 is configured to release to an open configuration, shown in FIG. 4B and 4C, when not confined by, for example, an outer sheath 412. In some embodiments, the deflector 460 is configured to fold along the central axis 464 away from the elongate member 462 so that the wings or petals 460a and 460b come together and the deflector 460 can be contained in, for example, an outer sheath 412, as shown in FIG. 4A. As shown in FIG. 4A, the deflector 460 can initially be folded and contained in the outer sheath 412 such that the wings or petals 460a and 460b are positioned distal to the central axis 464. In some embodiments, the deflector 460 can initially be folded in the opposite direction such that the wings or petals 460a and 460b are positioned proximal to the central axis 464.

In some embodiments, the catheter 402 is a pigtail-type catheter as shown in FIG. 4 and described herein. In some embodiments, the deflector 460 and embolic filter 410 can be coupled to another type of catheter, for example a catheter without a distal portion configured to assume an arcuate shape. The embolic filter 410 can be similar to the embolic filters 110 and 210 shown in FIGS. 1A-1D and 2A-2B and described herein. In some embodiments, the embolic filter 410 is coupled to the catheter 402 proximal to the deflector 460, for example as shown in FIG. 4A-4B. In some embodiments, the embolic filter 410 is coupled to the catheter 402 distal to the deflector 460. The embolic filter 410 is coupled so that it is disposed around the catheter 402. This configuration advantageously allows the embolic filter 410 to engage the interior body lumen 580 (see FIG. 5) wall, as the position of the catheter 402 within the body lumen 580 (see FIG. 5) may be affected by the deployed deflector 460.

The combination of the deflector 460 and the embolic filter 410 can advantageously provide additional protection against potential complications resulting from thrombi in the blood stream. For example, if the embolic filter 410 (e.g., the distal end of the embolic filter 410) is distal to the deflector 460, the embolic filter 410 can serve as the primary means of embolic protection and the deflector 460 can serve as the secondary means of embolic protection. If some blood is able to flow around the filter 410 rather than through it, the deflector 460 serves as a back-up protection device and prevents any debris not captured by the filter 410 from entering the cerebral arteries and traveling to the brain. If the embolic filter 410 is proximal to the deflector 460, the deflector 460 can serve as the primary means of embolic protection and the embolic filter 410 can serve as the secondary means of embolic protection. The deflector 460 first deflects debris away from the carotid arteries, then the embolic filter 410 captures debris (e.g., including deflected debris) as blood flows through the descending aorta.

In some embodiments, the catheter 402 and outer sheath 412 can have lips, shoulders, nubs, and/or detents, for example similar to those shown in FIGS. 2A-2B and described herein. For example, lips, shoulders, nubs, and/or detents can be positioned on the catheter 402 distal to the deflector 460, between the deflector 460 and embolic filter 410, and proximal to the embolic filter 410 to engage corresponding lips, shoulders, nubs, and/or detents on the outer sheath 412. The lips, shoulders, nubs, and/or detents can advantageously provide the user with information about the longitudinal position of the outer sheath 412 so that the user knows when neither, one, or both of the deflector 460 and embolic filter 410 are deployed. In some embodiments, either or both of the deflector 460 and embolic filter 410 can be movably coupled to the catheter 402 via an intermediate tube similar to that shown in FIGS. 3A-3D and described herein.

### III. Methods of Capturing Embolic Debris

FIGS. 5A-5D show one embodiment of a method of capturing embolic debris during a closed-heart medical procedure, for example an aortic valve replacement procedure. The method can be performed using, for example, an embolic protection device 100, 200, 300, or 400 as described herein.

In one embodiment, a guidewire 540 is percutaneously inserted into a body lumen 580 of a patient, for example a femoral, radial, brachial, or subclavian artery, and navigated to the desired anatomical location, for example, the level of the ascending aorta. The guidewire 540 can be a J tipped wire having a diameter of about 0.035 in. (approx. 0.089 cm). Other types and dimensions of guidewires 540 are also possible.

In some embodiments, the proximal end of the guidewire 540 is inserted into the opening at the distal end 116 of the catheter 102. When the guidewire 540 is in the lumen 118 of the catheter 102 at the distal portion 104 of the catheter 102, the distal portion 104 of the catheter is straightened or assumes the curvature of the guidewire 540. The distal end 116 of the catheter 102 is inserted into the body lumen 580 by tracking the lumen 118 of the catheter 102 over the guidewire 540, as shown in FIG. 5A. The outer diameter of the guidewire 540 is smaller than the inner diameter of the embolic protection device 100 such that the embolic protection device 100 may be tracked over the guidewire 540. The inner surface of the lumen 118 and/or the outer surface of the guidewire 540 may include a lubricious coating to reduce friction during tracking. The guidewire 540 keeps the distal portion 104 of the catheter 102 substantially straight (e.g., from being in the generally arcuate state) as the catheter 102 is inserted into and navigated within the patient's body.

The radiopaque marker 106 is used to visualize and position the distal portion 104 of the catheter 102 during tracking. The guidewire 540 is retracted, i.e., moved longitudinally in a proximal direction, a sufficient distance to allow the distal portion 104 of the catheter 102 to assume the generally arcuate shape, as shown in FIG. 5B. The distal portion 104 of the catheter 102 is positioned at the desired anatomical landmark, for example, the lower border of the noncoronary cusp of the aortic valve. The radiopaque marker 106 is on the distal-most section of the distal portion 104.

In some embodiments of the method, the proximal end 114 of the catheter 102 is connected to a contrast material injector, and contrast material is injected into the lumen 118 of the catheter 102, for example to visualize the anatomy around the device 100. The contrast material exits the catheter 102 lumen 118 through the opening at the distal end 116 of the catheter 102 and/or through one or more apertures 108 in the side wall of the catheter 102. Injecting contrast material can aid in visualizing and positioning the catheter 102.

In some embodiments, a second guidewire is percutaneously inserted into a second body lumen, for example the other femoral artery, and a second catheter is tracked over the second guidewire. The second catheter can carry a medical device or instrument, for example, a replacement valve, a valve repair system, or a radio frequency ablation system. Once the second catheter and associated device or instrument are properly positioned, the outer sheath 112 of the catheter 102 is longitudinally proximally retracted, allowing the embolic filter 110 to assume the expanded, deployed configuration, as shown in FIG. 5C. The second guidewire and/or the second catheter can also be positioned after the embolic filter 110 is released. The open distal end 140 of the embolic filter 110 is located in the ascending aorta so that blood flows through the filter before flowing into the carotid arteries or descending aorta. In some embodiments, when the embolic filter 110 is deployed, the catheter 102 rests against the interior lumen wall, thereby stabilizing the catheter 102. The procedure can then be performed, and embolic debris dislodged or otherwise in the blood stream during the procedure is captured by the embolic filter 110.

After the procedure, the outer sheath 112 is longitudinally distally advanced to recapture the embolic filter 110, returning the embolic filter 110 to the collapsed configuration and capturing any embolic debris 550 contained within the embolic filter 110, as shown in FIG. 5D. The second catheter and catheter 102 can then be withdrawn from the patient's body. The catheter 102 can be retracted over the guidewire 540 or without straightening the distal portion 104 of the catheter 102 because the arcuate shape of the distal portion 104 is atraumatic to the blood vessels.

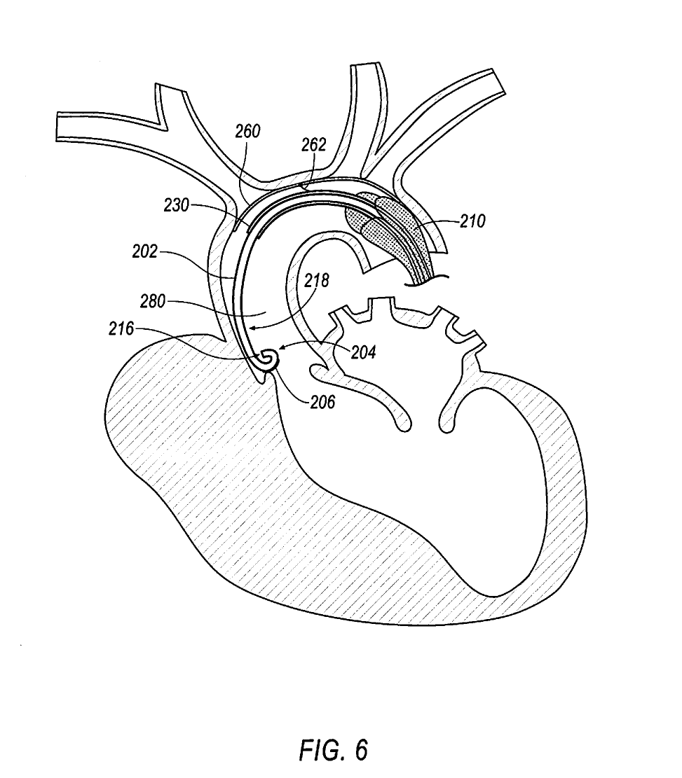

FIG. 6 illustrates another embodiment of a method of deflecting and capturing embolic debris during a medical procedure using an embolic protection device. The embolic protection device is similar to the embolic protection device 200 that is described in FIG. 2A-2D, wherein an intermediate tube 230 is longitudinally movable with respect to the catheter 202. Some embodiments employ a separate deflector device 562 of FIG. 5A-5B.

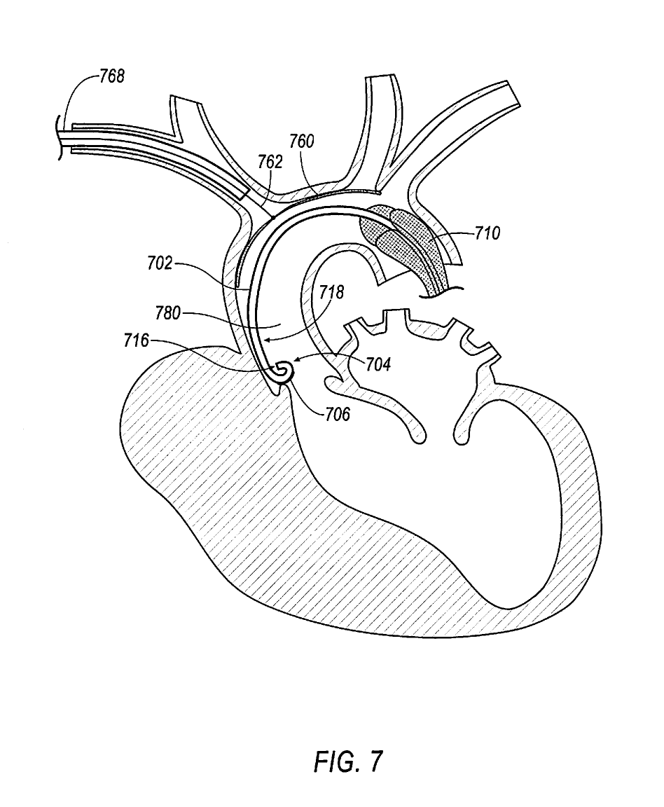

FIG. 7 illustrates another embodiment of deflecting and capturing embolic debris. An embolic protection device 700 of FIG. 7 comprises a catheter 702 (e.g., a pigtail catheter) with a radiopaque marker 706 and an embolic filter 710 disposed around the catheter 702 similar to embolic filter 410 illustrated in FIGS. 4A-4B and described herein. As shown, a deflector 760 is mounted to a shaft 762 and contained in an introducer 768 during insertion. The introducer 768 is introduced into the patient's body through the artery (e.g., right radial artery) and navigated to the aortic arch via the brachiocephalic artery. Once in position, the deflector 760 is deployed from the introducer and pulled back to cover the brachiocephalic and left common carotid artery. In some patients, the deflector 760 might also cover the left subclavian artery. In some embodiments, the deflector 760 can be introduced and deployed before the catheter 702 is navigated to the aortic arch. During a subsequent medical procedure, the deflector 760 can prevent emboli from entering the carotid arteries, and the embolic filter 710 can capture emboli deflected by the deflector 760 before it travels to other parts of the patient's body. The method can also be performed with various other embolic protection devices, for example as described herein, and deflector devices that may vary in configuration and how they are introduced into the body and navigated to the aortic arch.

In some embodiments, the procedure performed is a cardiac valve replacement procedure, for example an aortic valve replacement procedure. The embolic protection device 100 is introduced into the patient and navigated to the aortic valve as described herein and shown in FIGS. 5A-5D. The radiopaque marker 106 assists in delineating the lower border of the noncoronary cusp to assist in proper positioning of a percutaneously implanted replacement aortic valve. Once the catheter 102 is positioned, a second guidewire can be percutaneously inserted into a second body lumen and navigated to the level of the ascending aorta or left ventricle. A balloon can be tracked over the second guidewire to the aortic valve. The outer sheath 112 is then retracted to deploy the embolic filter 110. Balloon inflation of the valve can then be performed, and the embolic filter 110 captures embolic debris 550 dislodged during the procedure or otherwise in the blood stream. After balloon pre-dilation, the outer sheath 112 is advanced to recapture the embolic filter 110 and any embolic debris 550 contained within the embolic filter 110. The balloon is removed, and a second catheter carrying a valvular prosthesis is advanced to the level of the ascending aorta by tracking the catheter over the second guidewire. The outer sheath 112 is again retracted to redeploy the embolic filter 110. The radiopaque marker 106 allows the user to properly position the valve prosthesis, for example about 4 mm to about 6 mm below the lower border of the noncoronary cusp. After the procedure is completed, the outer sheath 112 is advanced to recapture the embolic filter 110 and any captured embolic debris 550, and the catheters are removed from the body. In some embodiments, the second catheter can be removed prior to advancing the outer sheath 112 to recapture the embolic filter 110 and embolic debris 550.

In some embodiments, the procedure is a cardiac valve repair procedure. The method described herein can also be adapted for a mitral valve repair or replacement procedure. In some embodiments, the procedure is a radio frequency ablation procedure, for example to treat atrial fibrillation. In some embodiments, the procedure is a catheterization procedure or structural heart procedure.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. An embolic protection device comprising:
a catheter having a proximal end, a distal end, and a lumen extending from the proximal end of the catheter to the distal end of the catheter, wherein the lumen is configured to house a guidewire, and a distal portion of the catheter assumes a generally arcuate shape being at least a semi-circle when the guidewire is at least partially longitudinally retracted;
a longitudinally-extending radiopaque marker located on at least a portion of the distal portion of the catheter that assumes the generally arcuate shape being at least a semi-circle;
a self-expanding embolic filter that is disposed around the catheter proximal to the distal portion, wherein the embolic filter comprises a frame that has at least two lobes, and the frame defines an opening of the embolic filter that faces the distal end of the catheter; and
a deployment mechanism that is disposed around at least a portion of the catheter, wherein the deployment mechanism is longitudinally movable with respect to the catheter, the deployment mechanism is configured to contain the embolic filter in a collapsed configuration, and the embolic filter is configured to self-expand upon the longitudinal retraction of the deployment mechanism.

2. The embolic protection device of claim 1, wherein the embolic filter comprises a filter medium that attaches to at least a portion of the frame.

3. The embolic protection device of either of claim 1 or 2, wherein the frame comprises a shape memory material.

4. The embolic protection device of claim 3, wherein the shape memory comprising a metal alloy or polymer.

5. The embolic protection device of claim 4, wherein the shape memory material comprises a metal alloy.

6. The embolic protection device of claim 5, wherein the alloy comprises nitinol or chromium colbalt.

7. The embolic protection device of claim 5, wherein the alloy is selected from MP35N, 35NLT, and Elgiloy.

8. The embolic protection device of any one of claims 1-7, wherein the filter medium comprises a woven or non-woven material.

9. The embolic protection device of any one of claims 1-7, wherein the filter medium comprises a semi-permeable polyurethane material having a pore size of from about 100 microns to about 150 microns.

10. The embolic protection device of any one of claims 1-9, wherein the embolic filter is movably coupled to the catheter and is longitudinally moveable with respect to the catheter.

11. The embolic protection device of any one of claims 1-10, further comprising a self-expanding deflector coupled to the catheter proximal to the distal portion, wherein the deflector has a longitudinal axis parallel to the longitudinal axis of the catheter.

12. The embolic protection device of any one of claims 1-11, wherein the deployment mechanism comprises a sheath that is circumferentially disposed around at least a portion of the catheter, wherein the sheath deploys the self-expanding embolic filter when the sheath is at least partially longitudinally retracted.

13. The embolic protection device of claim 12, wherein the sheath comprises a polymer material.

14. The embolic protection device of any one of claims 1-13, wherein the distal portion of the catheter comprises one or more apertures that communicates with the lumen of the catheter.

15. A method of capturing embolic debris during a closed-heart procedure, the method comprising:
inserting a distal end of a embolic protection device into a body lumen by tracking a lumen of the catheter over a guidewire that is percutaneously inserted into the body lumen, the embolic protection device comprising:
a catheter having a proximal end, a distal end, and a lumen extending from the proximal end of the catheter to the distal end of the catheter, wherein the lumen is configured to house a guidewire, and a distal portion of the catheter assumes a generally arcuate shape being at least a semi-circle when the guidewire is at least partially longitudinally retracted;
a longitudinally-extending radiopaque marker located on at least a portion of the distal portion of the catheter that assumes the generally arcuate shape being at least a semi-circle;
a self-expanding embolic filter that is disposed around the catheter proximal to the distal portion, wherein the embolic filter comprises a frame that has at least two lobes, and the frame defines an opening of the embolic filter that faces the distal end of the catheter when the embolic filter is deployed; and
a deployment mechanism that is disposed around at least a portion of the catheter, wherein the deployment mechanism is longitudinally movable with respect to the catheter, the deployment mechanism is configured to contain the embolic filter in a collapsed configuration, and the embolic filter is configured to self-expand upon longitudinal retraction of the deployment mechanism.

16. The method of claim 15, further comprising at least partially longitudinally retracting the guidewire from the lumen of the catheter, so that the distal portion of the catheter assumes a generally arcuate shape being at least a semi-circle.

17. The method of either of claim 15 or 16, further comprising positioning the catheter by visualizing the radiopaque marker using an imaging technique.

18. The method of any one of claims 15-17, further comprising at least partially longitudinally retracting the deployment mechanism and allowing the self-expanding embolic filter to assume an expanded, deployed configuration having an distal opening defined by the frame that substantially spans the body lumen.

19. The method of any one of claims 15-18, wherein the embolic filter comprises a filter medium that attaches to at least a portion of the frame.

20. The method of any one of claims 15-19, wherein the frame comprises a shape memory material.

21. The method of claim 20, wherein the frame comprises a shape memory material comprising a metal alloy or polymer.

22. The method of claim 21, wherein the shape memory material comprises a metal alloy comprising nitinol or chromium cobalt.

23. The method of claim 21, wherein the shape memory material comprises a metal alloy selected from MP35N, 35NLT, and elgiloy.

24. The method of any one of claims 15-19, wherein the embolic filter comprises a filter medium comprising a woven or non-woven material.

25. The method of claim 24, wherein the filter medium comprises a semi-permeable polyurethane material having a pore size of from about 100 microns to about 150 microns.

26. The method of any one of claims 15-25, wherein the embolic filter is movably coupled to the catheter and is longitudinally moveable with respect to the catheter.

27. The method of any one of claims 15-26, wherein the embolic protection device further comprises a self-expanding deflector coupled to the catheter proximal to the distal portion, wherein the deflector has a longitudinal axis parallel to the longitudinal axis of the catheter.

28. The method of any one of claims 15-27, wherein the deployment mechanism comprises a sheath that is circumferentially disposed around at least a portion of the catheter, wherein the sheath deploys the self-expanding embolic filter when the sheath is at least partially longitudinally retracted.

29. The method of claim 28, wherein the sheath comprises a polymer material.

30. The method of any one of claims 15-29, wherein the distal portion of the catheter comprises one or more apertures that communicates with the lumen of the catheter.

31. The method of claim 30, further comprising perfusing a fluid into the body lumen through the one or more apertures.

32. A method of capturing embolic debris during a closed heart procedure, the method comprising:
inserting a distal end of a embolic protection device into a body lumen by tracking a lumen of the catheter over a guidewire percutaneously inserted into the body lumen, the embolic protection device comprising:
a catheter having a proximal end, a distal end, and a lumen extending from the proximal end of the catheter to the distal end of the catheter, the lumen configured to house a guidewire, a distal portion of the catheter to assume a generally arcuate shape being at least a semi-circle;
the distal portion of the catheter comprising a longitudinally-extending radiopaque marker located on at least a portion of the distal portion of the catheter that assumes the generally arcuate shape being at least a semi-circle;
a self-expanding embolic filter that is disposed around the catheter proximal to the distal portion, wherein the embolic filter comprises a frame that has at least two lobes, and the frame defines an opening of the embolic filter that faces the distal end of the catheter when the embolic filter is deployed; and
a deployment mechanism that is disposed around at least a portion of the catheter, wherein the deployment mechanism is longitudinally movable with respect to the catheter, the deployment mechanism is configured to contain the embolic filter in a collapsed configuration, and the embolic filter is configured to self-expand upon longitudinal retraction of the deployment mechanism;
at least partially longitudinally retracting the guidewire from the lumen of the catheter, so that the distal portion of the catheter assumes a generally arcuate shape being at least a semi-circle upon retracting the guidewire from the distal portion of the catheter;
positioning the catheter by visualizing the radiopaque marker using an imaging technique;
longitudinally retracting the deployment mechanism and deploying the self-expanding embolic filter, so that the embolic filter assumes an expanded, deployed configuration, wherein the opening defined by the frame substantially spans the body lumen.
